(19) United States
(12) Patent Application Publication
(10) Pub. No.: US 2008/0025970 A1
(43) Pub. Date: Jan. 31, 2008

(54) Treatment of atherosclerosis

(76) Inventor: Robin N. Poston, London (GB)

Correspondence Address: ROTHWELL, FIGG, ERNST & MANBECK, P.C., 1425 K STREET, N.W., SUITE 800, WASHINGTON, DC, 20005, US

(21) Appl. No.: 11/663,588

(22) PCT Filed: Sep. 22, 2005
(86) PCT No.: PCT/GB05/03669

§ 371 Date: Jun. 1, 2007

(30) Foreign Application Priority Data
Sep. 23, 2004 (GB) 0421176.9

(51) Int. Cl. A61K 39/395; A61K 31/335; A61K 31/713; A61P 9/10; G01N 33/569; A61K 31/715; A61K 36/062

(52) U.S. Cl. 424/130.1; 424/195.15; 435/7.1; 514/44; 514/450; 514/58

(57) ABSTRACT

Inhibitors of the adhesion of monocytes to oxidised low density lipoprotein or other lipid raft ligand for the therapy or prophylaxis of a condition involving monocyte adhesion to oxidised LDL or other lipid raft ligand, such as atherosclerosis, rheumatoid arthritis, multiple sclerosis or glomerulonephritis. An assay for identifying such inhibitors.

### FIELD OF INVENTION

The present invention relates to the treatment of atherosclerosis. More part atherosclerosis and an assay for identifying such inhibitors. Further, the invention relates to inhibitors of lipid raft mediated monocyte adhesion for the treatment of atherosclerosis.

### BACKGROUND TO THE INVENTION

Atherosclerosis is the underlying disease process responsible for vascular conditions that causes the death of over one third of the population of the Western world. The adhesion of blood monocytes to the wall of susceptible arteries, and their subsequent migration into the wall, are now accepted stages in the pathogenesis of atherosclerosis. Analogy with inflammation and the identity of the distribution of this cellular traffic with the focal occurrence of lesions in the arterial tree are evidence that these monocyte events are critical regulatory stages in the development of the disease. In both atherosclerosis and inflammation, there is focal increased expression of endothelial adhesion molecules in the affected areas. Multiple molecules have been identified as active in inducing monocyte adhesion, and initially similar molecules such as endothelial ICAM-1 binding to monocyte β₂ integrins were implicated in both conditions¹.

Considerable work has been directed at elucidating adhesion mechanisms. Beekhuizen and van Furth (1993)² showed that the adhesion of monocytes to cultured human umbilical vein endothelial cells (HUVEC) was substantially dependent on monocyte CD14. This led to the demonstration by Poston et al (1996)³ that the adhesion of monocytes to the endothelium of tissue sections of human atherosclerotic arteries in vitro was similarly CD14 dependent. CD14 is well known as a receptor for bacterial endotoxin involved in innate immunity, and CD14 and multiple signaling molecules and co-receptors such as toll-like receptor 4 (TLR-4) are aggregated together in cholesterol and GM1 ganglioside-rich membrane sub-domains, termed lipid rafts^{4,5}.

Although the role of lipid rafts in adhesion has not been precisely defined, they are clearly involved, e.g. integrins aggregate and locate to them with cell activation^{6,7}. Recently a potential endogenous ligand of CD14 came to light, heat shock protein 60 (HSP60)⁸. HSP60 can be expressed in stressed endothelial cells (ECs), including those activated with TNFα⁹. It has recently been confirmed that HSP60 is expressed on the surface of HUVEC after TNF stimulation. Furthermore, recent work suggested that HSP60 has a role in monocyte migration into human atherosclerotic lesions, as the adhesion of PMA-stimulated U937 cells to the endothelium of plaques in tissue sections is inhibited by antibodies to HSP60^{10,11}. Further, it was found that both PMA differentiated U937 cells, and isolated blood monocytes will bind to solid phase HSP60 in a CD14 dependent manner in a static adhesion assay.

Lipid rafts are labile structures modulated by ligand-receptor interactions, cell signaling and the like. Lipid rafts on monocytes/macrophages provide a dynamic microenvironment for an integrated CD14-dependent clustering of a set of receptors involved in inflammation and atherogenesis⁴. Ligand binding promotes conformational changes and specific co-assembly of additional receptors to the basal cluster present on resting cells. The composition of the receptor cluster and thus the associated signaling pathways define a ligand-specific cellular response.

It has also been shown that in ligation of endotoxin or HSP60, intracellular signaling pathways are activated via TLR-4¹². The signaling that follows is complex and mediated by multiple factors, including the adaptor protein MyD88, the interleukin-1 associated kinase, p38 MAP kinase, and the G protein Rap1^{13,14}, a pathway leading to integrin activation. Thus an additional adhesion mechanism with CD14 ligands may be that intracellular signaling allows activation of integrins. In a recent study on mouse atherosclerosis, deficiency of the signaling protein MyD88, but not of CD14, was shown to reduce disease¹⁵. MyD88 mediates signaling from toll-like receptors in lipid rafts, and from the non-raft-associated cytokines IL-1 and IL-18.

A further CD14 ligand of direct relevance to atherosclerosis has been identified by Miller et al (2003)^{16,17}, who showed that macrophages can have their motility modified by CD14 dependent binding to oxidised LDL.

It is clear that the adhesion and migration of leukocytes, and particularly monocytes, to the arterial wall plays an important role in the development of atherosclerotic lesions and, in fact, this is probably the critical rate-limiting factor in their development. For this reason, there is a need for further identification of the interactions that are important in the adhesion and migration of monocytes in atherosclerotic lesions.

The involvement of oxidised low density lipoprotein (LDL) in atherosclerosis is already known. There is much oxidised LDL extracellularly within atherosclerotic lesions, where it is phagocytosed and internalised by macrophages. Minimally modified LDL produced by mild oxidation of LDL is a pro-inflammatory and pro-atherogenic lipoprotein that is recognised by the LDL receptor but is not recognised by macrophage scavenger receptors and thus does not have enhanced uptake by macrophages. Miller et al¹⁶ showed that minimally modified LDL binds to CD14 on macrophages, induces actin polymerisation and macrophage spreading via TLR-4/MD-2, which results in such pro-atherogenic consequences as inhibition of phagocytosis of apoptotic cells and enhancement of oxidised LDL uptake. Miller et al do not present data on monocytes or cellular adhesion.

Several publications teach that incubation of endothelial cells with oxidised or otherwise modified LDL enhances monocyte adhesion. Frostegard et al¹⁸, Jeng et al¹⁹, and Klouche et al²⁰ consider that it is operating by stimulation of conventional adhesion mechanisms, but none suggest that it is itself a ligand for monocyte binding. The most recent by Dwivedi et al (2001)²¹ shows that the endothelial cell line EAHy926, when incubated with 100 μg/ml of oxidised LDL, induces over 75% adhesion of human peripheral blood monocytes, a response equal to that by the classic stimulator TNFα. They consider however that this response is atypical for involvement of conventional adhesion mechanisms, as the transcription factor NFkB is not induced, and the levels of the adhesion molecules ICAM-1 and VCAM-1 are not increased.

### SUMMARY OF THE INVENTION

It has now been found that monocytes adhere to oxidised low density lipoprotein (LDL) or components thereof. Furthermore it has been found that the arterial endothelium of atherosclerotic human arteries contains oxidised LDL, whilst components of oxidised LDL, such as oxidised apoproteins, oxidised phospholipids and oxidised neutral lipids, may be present individually on the surfaces of cells such as endothelial cells. It is therefore proposed that monocytes bind to the arterial wall by interacting directly with oxidised LDL or components thereof, in addition to binding through conventional adhesion molecules. This adhesion may play a major part in the adhesion of monocytes to atherosclerotic lesions, so may have particular application in the therapy of atherosclerosis.

It has also been found that lipid rafts or microdomains on monocytes are involved in the adhesion process, again in a manner which is involved in atherosclerosis but may have less importance in other inflammatory processes. Therefore the destabilization of monocyte lipid rafts may provide a novel powerful means of regulating cellular adhesion. As these rafts can bind to multiple ligands with potential roles in atherosclerosis such as integrin ligands, oxidised LDL, and HSP60, their modulation may have application in the therapy of atherosclerosis.

Investigation of a class A scavenger receptor inhibitor and an integrin inhibitor suggest a role for these receptors in adhesion of monocytes to oxidised LDL. Further, an extensive panel of signaling inhibitors all markedly reduced adhesion. These results suggest that the components on which they act are all involved in an integrated manner in allowing raft adhesive function in monocytes, permitting a high level of adhesion to oxidised LDL. This is an analogous mechanism to that involved in the adhesion of T lymphocytes to antigen presenting cells, in which the lipid raft structure involved is termed the immunological synapse²².

When investigating the adhesion of highly activated U937 cells to oxidised LDL and HSP60, it was found unexpectedly that non-immune immunoglobulins (Igs) were highly inhibitory. This did not occur with U937 cells treated with PMA alone, or with monocytes isolated without activation under endotoxin-free conditions. This is evidence that receptors for Igs in the rafts, probably the Fc receptors CD16, CD32 and/or CD64, have a role in inhibiting the adhesive function of adhesion receptors in the rafts. Thus Igs or other ligands binding to Fc receptors provide a route for the inhibition of raft mediated adhesion of activated monocytic cells.

Monocytes in the circulation when activated would be expected to have adhesion function inhibited by the high concentrations of Igs present; however if they migrated into tissues containing less Ig, the adhesive activity of their rafts could be revealed. In that instance, a low MW agonist of Ig receptors could be effective in inhibiting monocyte/macrophage adhesion and thence disease processes resulting.

Although the invention relates particularly to monocyte adhesion to oxidised LDL or a component thereof, adhesion of highly activated U937 cells to fibronectin was also inhibited by immunoglobulins, most probably through inhibition of lipid raft function. Fibronectin is a ligand for the major integrin family of adhesion molecules. Hence a wide variety of diseases mediated by adhesive monocytes could be treated by Igs or related inhibitors of lipid raft function, preferably by low molecular weight ligands at Ig receptors, preferably at the Fc receptors CD16, CD32 and CD64. Diseases mediated by adhesive monocytes include rheumatoid arthritis, glomerulonephritis and multiple sclerosis.

Although oxidised LDL and its components are likely to be generated in large quantities in the endothelial cells and related parts of atherosclerotic lesions, it is possible that endothelial cells elsewhere may take up and oxidise LDL, and the oxidised LDL formed contributes to monocyte adhesion in the target organ. Therefore inhibitors of adhesion of monocytes to oxidised LDL or components thereof, preferably low molecular weight inhibitors, may offer a route to the therapy of a wide range of diseases mediated by adhesive monocytes.

Accordingly, in a first aspect the present invention provides a method for identifying agents which inhibit binding of monocytes to oxidised LDL or a component thereof which comprises:
contacting a suspension of monocytes with immobilised oxidised LDL or a component thereof, in the presence and absence of a potential inhibitory agent, or with and without preincubation of the monocyte suspension or immobilised oxidised LDL or component thereof with a potential inhibitory agent;
quantitating the number of bound monocytes over a defined area of oxidised LDL or component thereof; and
assessing the effect of the agent on the extent of monocyte binding, wherein agents which inhibit binding of monocytes to oxidised LDL or component thereof are identified by a reduction in the number of bound monocytes over the defined area.

Preferably the agent which inhibits binding of monocytes to oxidised LDL is identified by a 50% reduction in the number of bound monocytes over the defined area. Preferably the oxidised LDL is immobilised on the bottom surface of a plastic plate. The component of oxidised LDL may be an oxidised apoprotein, oxidised phospholipid or oxidised neutral lipid.

In another aspect, the invention provides a method for the inhibition of monocyte adhesion to oxidised LDL or component thereof in a patient which comprises administering to the patient an effective amount of an agent which inhibits said monocyte adhesion to oxidised LDL or component thereof. Preferably the agent produces a reduction of at least 50% in the number of bound monocytes in the method as described above. Preferably said inhibition of monocyte adhesion to oxidised LDL or component thereof is for the treatment of atherosclerosis, rheumatoid arthritis, multiple sclerosis or glomerulonephritis. The agent may comprise nystatin, methyl cyclodextrin, dimethylsulphoxide, or combinations or derivatives thereof. Alternatively, the agent may comprise an immunoglobulin, a signaling pathway inhibitor, or a ligand at monocyte immunoglobulin receptors, preferably at the lipid raft-related Fc receptors CD16, CD32 or CD64. A low molecular weight agonist or chemical molecule is preferred, for example molecules with a molecular weight up to about 1000.

In a further aspect, the invention provides a method for the inhibition of lipid raft mediated monocyte adhesion in a patient which comprises administering to the patient an effective amount of an agent which inhibits said lipid raft mediated monocyte adhesion. Such an agent may also inhibit the binding of monocytes to oxidised LDL or a component thereof, and preferably the agent produces a reduction of at least 50% in the number of bound monocytes in the method as described above. Preferably said inhibition of lipid raft mediated monocyte adhesion is for the treatment of atherosclerosis, rheumatoid arthritis, multiple sclerosis or glomerulonephritis. The agent may comprise nystatin, methyl cyclodextrin, dimethylsulphoxide, or combinations or derivatives thereof. Alternatively, the agent may comprise an immunoglobulin, a signaling pathway inhibitor, or a ligand at monocyte immunoglobulin receptors, preferably at the lipid raft-related Fc receptors CD16, CD32 or CD64. A low molecular weight agonist or chemical molecule is preferred, for example molecules with a molecular weight up to about 1000.

In yet another aspect, the invention relates to the use of an agent that inhibits the adhesion of monocytes to oxidised LDL or component thereof for the manufacture of a medicament for the therapy or prophylaxis of a condition involving monocyte adhesion to oxidised LDL or component thereof by a method wherein said monocyte adhesion to oxidised LDL or component thereof is inhibited. Preferably the agent produces a reduction of at least 50% in the number of bound monocytes in the method as described above. Preferably said inhibition of monocyte adhesion to oxidised LDL or component thereof is for the treatment of atherosclerosis, rheumatoid arthritis, multiple sclerosis or glomerulonephritis. The agent may comprise nystatin, methyl cyclodextrin, dimethylsulphoxide, or combinations or derivatives thereof. Alternatively, the agent may comprise an immunoglobulin, a signaling pathway inhibitor, or a ligand at monocyte immunoglobulin receptors, preferably at the lipid raft-related Fc receptors CD16, CD32 or CD64. A low molecular weight agonist or chemical molecule is preferred, for example molecules with a molecular weight up to about 1000.

In a further aspect, the invention provides the use of an agent that inhibits lipid raft mediated monocyte adhesion for the manufacture of a medicament for the therapy or prophylaxis of a condition involving lipid raft mediated monocyte adhesion by a method wherein said lipid raft mediated monocyte adhesion is inhibited. Such an agent may also inhibit the binding of monocytes to oxidised LDL or a component thereof, and preferably the agent produces a reduction of at least 50% in the number of bound monocytes in the method as described above. Preferably said inhibition of lipid raft mediated monocyte adhesion is for the treatment of atherosclerosis, rheumatoid arthritis, multiple sclerosis or glomerulonephritis. The agent may comprise nystatin, methyl cyclodextrin, dimethylsulphoxide, or combinations or derivatives thereof. Alternatively, the agent may comprise an immunoglobulin, a signaling pathway inhibitor, or a ligand at monocyte immunoglobulin receptors, preferably at the lipid raft-related Fc receptors CD16, CD32 or CD64. A low molecular weight agonist or chemical molecule is preferred, for example molecules with a molecular weight up to about 1000.

The invention will now be described in more detail with reference to the figures in which:

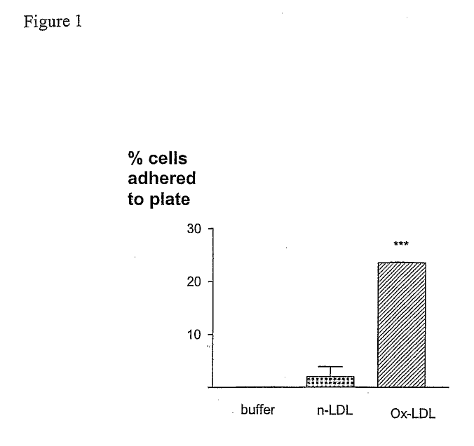

FIG. 1 shows the adhesion of PMA stimulated U937 cells to native and oxidised LDL;

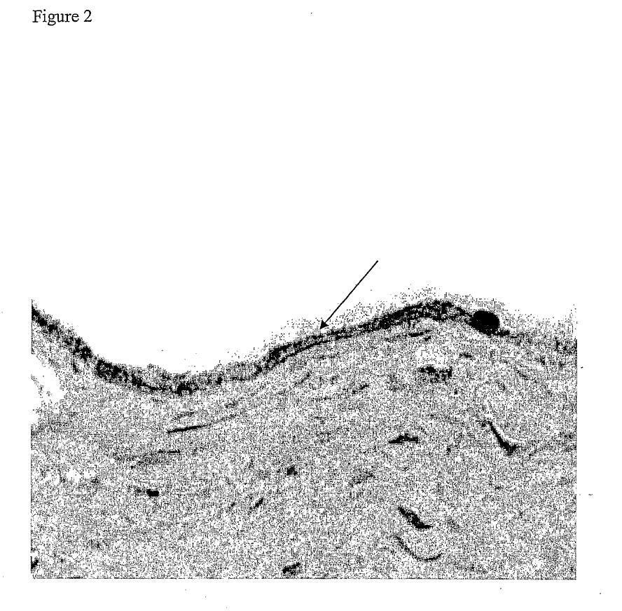

FIG. 2 shows that oxidised LDL is specifically localised in the endothelial cells over atherosclerotic lesions;

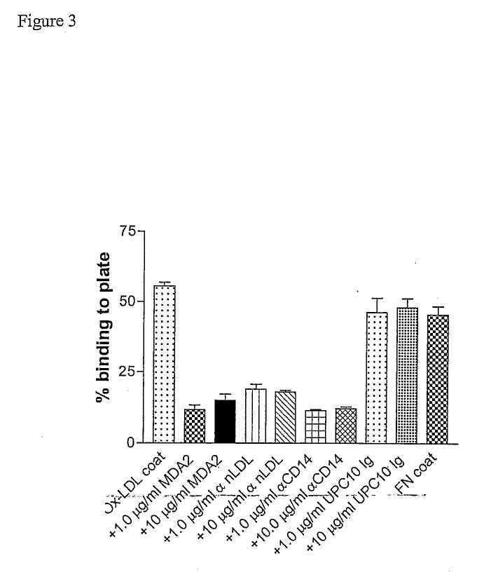

FIG. 3 shows the binding of monocytes to oxidised LDL in the presence of antibody inhibitors;

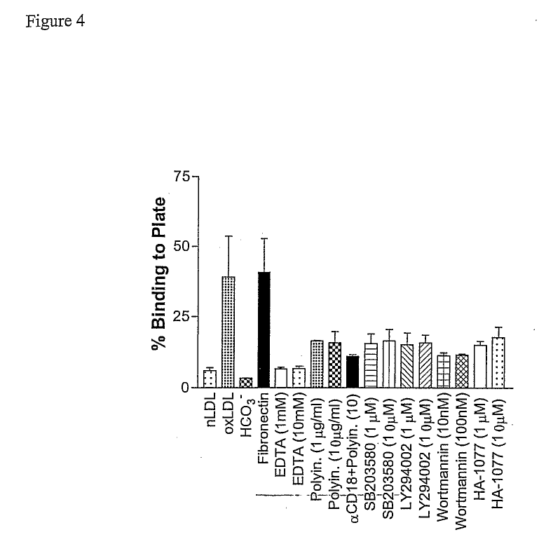

FIG. 4 shows the binding of monocytes to oxidised LDL in the presence of metabolic inhibitors;

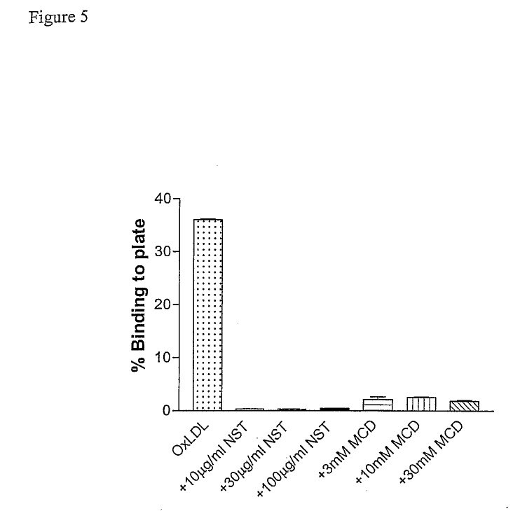

FIG. 5 shows that lipid raft disruption inhibits the adhesion of monocytes to oxidised LDL;

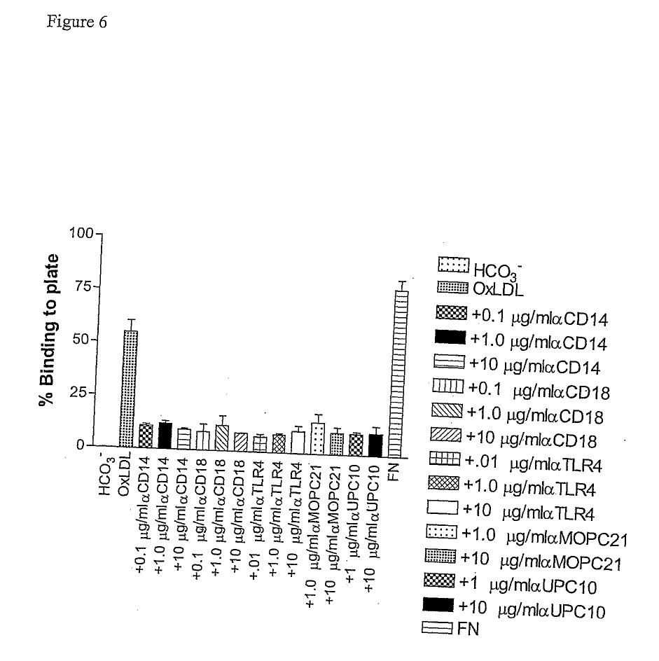

FIG. 6 shows the inhibition of binding of highly activated U937 cells to oxidised LDL by murine immunoglobulins;

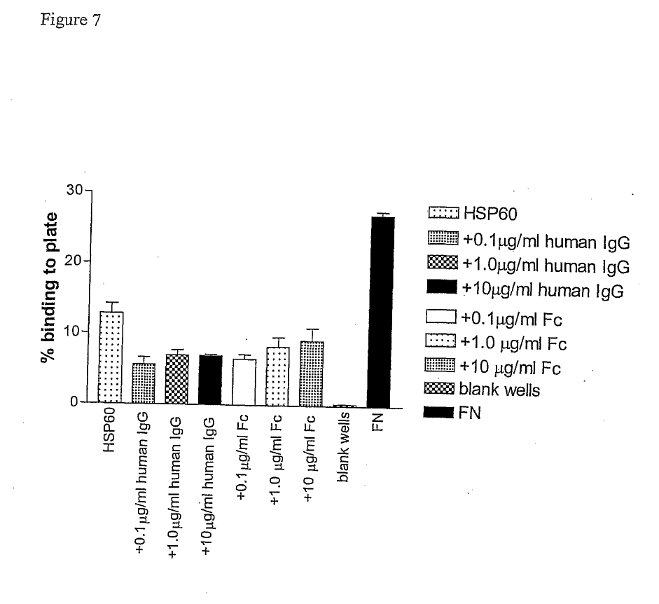

FIG. 7 shows the binding of highly activated U937 cells to HSP60 in the presence of Human IgG fragments;

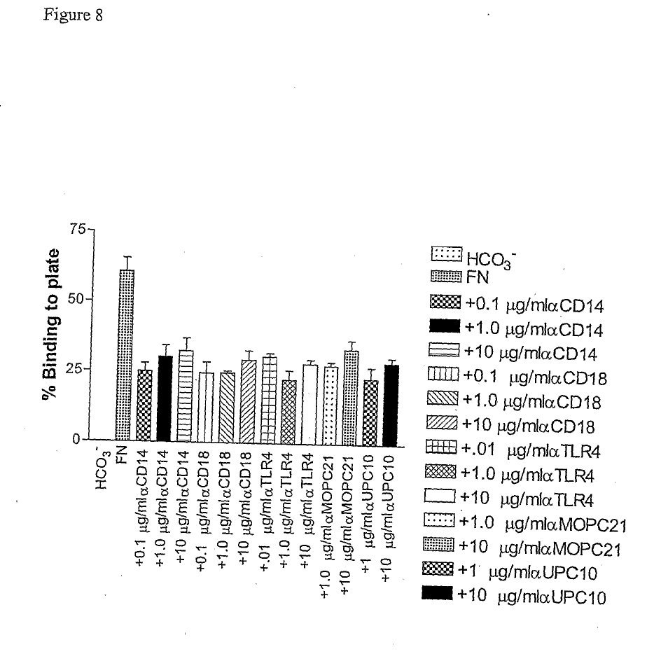

FIG. 8 shows the inhibition of the binding of highly activated U937 cells to fibronectin by murine immunoglobulins; and

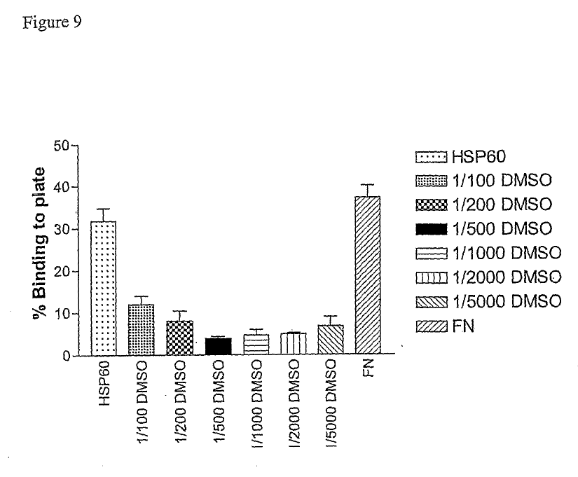

FIG. 9 shows the inhibition of adhesion of highly activated U937 cells to HSP60 by DMSO.

### DETAILED DESCRIPTION OF THE INVENTION

The present invention relates to inhibitors of the adhesion of monocytes to oxidised low density lipoprotein or its components for the therapy or prophylaxis of a condition involving monocyte adhesion to oxidised LDL, such as atherosclerosis, and an assay for identifying such inhibitors. Further, the invention relates to inhibitors of lipid raft mediated monocyte adhesion for the therapy or prophylaxis of a condition involving lipid raft mediated monocyte adhesion. It is a disadvantage of anti-adhesion therapies in general that they might inhibit useful inflammation and predispose to infection. Since this adhesion may play a major part in the adhesion of monocytes to atherosclerotic lesions, it may have particular application in the therapy of atherosclerosis. In particular, inhibitors of the adhesion of monocytes to oxidised low density lipoprotein, its components or other lipid raft ligand may be used for the treatment of atherosclerosis without inhibiting useful inflammation and predisposing the patient to infection.

As used herein the term “oxidised LDL” is understood to refer to components of oxidised LDL as well as to oxidised LDL per se. For example, oxidised components of LDL present individually on cell surfaces also adhere to monocytes and may contribute to the adhesion of monocytes to atherosclerotic lesions. In particular, oxidised phospholipids products such as 1-palmitoyl-2-(5′-oxo-valeroyl)-sn-glycero-3-phosphocholine (POVPC), derived from phospholipids contained in LDL such as 1-palmitoyl-2-arachidonyl-sn-glycero-3-phosphocholine (PAPC) are present on the cell surface of endothelial cells, while the whole ox-LDL particle may not be. They may incorporate themselves into the cell membrane. Other components behaving similarly are oxidised apoproteins and oxidised neutral lipids.

Any monocyte-like cell line can used in the method for identifying agents which inhibit binding of monocytes to oxidised LDL or a component thereof according to the invention. A monocyte-like cell line is a cell line which has adhesion properties similar to human monocytes so that it adheres to vascular tissue (for example arterial wall) in a similar manner to human monocytes. The adhesion properties of monocytes are, in turn, determined by the adhesion receptors on the surface of the cell.

The monocyte-like cell line is preferably a monoclonal cell line. One particularly preferred monocyte-like cell line is the U937 histiocytic lymphoma cell line according to Harris & Ralph (1985)²³ available to the public from ATCC number CRL 1593. The U937 cell line was first described by Sundstrom and Nilsson (1976)²⁴. An alternative monocyte-like cell line is the THP-1 monocyte cell line available to the public from ATCC number TIB 202 (see Tsuchiya et al (1980)²⁵).

The above monoclonal monocyte-like cell lines can be grown by standard methods in cell culture medium such as RPMI medium and will generally be used according to the invention in suspension in that or a similar medium. In the case of the U937 cell line, a preferred cell culture medium is RPMI medium containing 10% fetal calf serum and this medium can also be used for the assay but preferably containing 10 mM HEPES buffer. The monoclonal monocyte-like cells are preferably activated in order to increase adhesion, for example by use of a phorbol ester. According to one embodiment of the invention U937 cells can be activated by use of phorbol myristyl acetate (PMA), for example suspension in tissue culture medium containing 10 ng/ml phorbol myristyl acetate for 24-48 hours at 37° C.

Whilst monoclonal cell lines are preferred, normal human monocytes can also be used in the application of the method according to the invention. Normal human monocytes can be prepared from human blood by centrifuging on a Nycomed density gradient as described by Tsouknos et al (2003)²⁶. Alternatively, monocytes can be isolated from blood in an elutriation apparatus. Furthermore, by use of monocytes derived from patients' blood, the assay can also be employed to assess the adhesive properties of monocytes in patients with atherosclerotic or other disease.

LDL may be obtained from human serum by density gradient centrifugation, and oxidised with copper sulphate 5 μmol/L, as described by Siow et al (1998)²⁷. The oxidised LDL may immobilised in various ways, but immobilisation on the bottom surface of plastic plates is particularly preferred. Black 96 well plates (Corning) are optimally coated overnight at 4° C. with 50 μL of 10 μg/ml of oxidised LDL or isolated native LDL. Uncoated wells, or bovine serum albumin at the same concentration are used as negative controls. After washing, for example three times with PBS, the wells may be used in the adhesion experiment.

Preferably, U937 cells are stimulated overnight with PMA as above, and washed three times in RPMI+10% FCS. The U937 cells or monocytes are then incubated with 5 (and 6)-carboxyfluorescein diacetate succinimidyl ester (C-1157, Molecular Probes), which allows fluorescein labelling of viable cells. The cells are washed in RPMI+10% FCS, and added to the immobilised oxidised LDL. A suitable concentration of cells for contacting with the immobilised oxidised LDL is about 10⁵ to 10⁷ cells per ml, preferably about 3×10⁶ cells per ml, Inhibitors may also be added to the wells. Assays are ideally done at least in triplicate.

The immobilised oxidised LDL is contacted with the monocyte-like cell suspension under conditions and for a sufficient length of time that allow the cells to adhere to the immobilised oxidised LDL where suitable adhesion molecules are present to bring about such adhesion.

Preferably the assay according to the present invention is be carried out at a temperature of at least 10° C., for example about 15 to 45° C., preferably about 20 to 40° C., more preferably about 37° C. The plates are incubated for 40 minutes or 1 hour or longer, preferably about 40 minutes.

The plates are then washed by shaking out the cells and washing in PBS. Aliquots of the original cell suspension are added to at least three unused wells as reference. The plates are then counted in an automatic fluorescent spectrophotometer. The data are expressed as the % of input cells bound ±SD, compared to the reference wells.

The method according to the present invention is valuable for a number of purposes. The involvement of adhesion molecules in the entry of monocytes into atherosclerotic foci may be of profound significance as it appears to be a vital mechanism in this initial event in the generation of the disease. There is reason to suppose that once monocyte entry has started, it may be self-perpetuating, as factors produced by the monocyte-derived macrophages may elicit farther formation of endothelial adhesion molecules.

The method according to the present invention can be used for screening possible inhibitory agents with the potential for the development of therapeutic approaches against human atherosclerosis. Assaying for adhesion of monocyte-like cells to immobilised oxidised LDL or an immobilised component of oxidised LDL in the presence and the absence of a potential inhibitory agent will identify those agents which inhibit the adhesion process. Alternatively the cell suspension or the immobilised oxidised LDL or component thereof can be preincubated with a potential inhibitory agent. The assay can be performed in multi-well plates and is suitable for high-throughput screening of chemical libraries, for example for the discovery of active agents.

Accordingly, the present invention also relates to a method for the inhibition of monocyte adhesion to oxidised LDL or component thereof in a patient which comprises administering to the patient an effective amount of an agent which inhibits said monocyte adhesion to oxidised LDL or component thereof, for example for the treatment or prophylaxis of atherosclerosis, rheumatoid arthritis, multiple sclerosis or glomerulonephritis.

The present invention also relates to a method for the inhibition of lipid raft mediated monocyte adhesion in a patient which comprises administering to the patient an effective amount of an agent which inhibits said lipid raft mediated monocyte adhesion. Lipid rafts can bind to multiple ligands with potential roles in atherosclerosis such as integrin ligands, oxidised LDL, and HSP60. Consequently the modulation of lipid rafts by disruption of such ligand interactions may have potential for the therapy of atherosclerosis, rheumatoid arthritis, multiple sclerosis or glomerulonephritis. Further lipid rafts require acylation of proteins for them to enter²⁸, and this could be an additional target to the prenylation inhibited in the anti-inflammatory effect of statins.

Suitable agents for use in these methods may be identified using the assay described above; in particular, agents that inhibit lipid raft mediated monocyte adhesion may also inhibit the binding of monocytes to oxidised LDL or a component thereof. Thus an agent suitable for use in a method for the inhibition of monocyte adhesion to oxidised LDL may also be suitable for use in a method for the inhibition of lipid raft mediated monocyte adhesion.

Suitable agents for use in these methods include antibodies against oxidised LDL, native LDL or CD14 and other molecules, e.g. small chemical molecules, which inhibit monocyte adhesion to oxidised LDL. It may be possible to use rodent antibodies against oxidised LDL, native LDL or CD14. Other antibodies against oxidised LDL, native LDL or CD14 are available or can be derived using known methods. However, it is preferred to develop antibodies, preferably anti-oxidised LDL antibodies, which have less potential for eliciting a reaction from the human immune system using known techniques such as the production of chimeric or humanised (e.g. CDR grafted) antibodies.

It is more preferred in a therapeutic context to use small chemical molecules, for example molecules with a molecular weight up to about 1000. Other suitable inhibitory agents include nystatin, methyl cyclodextrin, dimethylsulphoxide, or combinations or derivatives thereof. Alternatively, the agent may comprise an immunoglobulin, a signaling pathway inhibitor, or a ligand at monocyte immunoglobulin receptors, preferably at the lipid raft-related Fc receptors CD16, CD32 or CD64. Preferably, the agent is polyinosine, SB203580, LB294002, wortmannin, HA-1077, or combinations or derivatives thereof

A medicament comprising an agent which inhibits the adhesion of monocytes to oxidised LDL, a component thereof or other lipid raft ligand may consist solely of the agent as the raw substance, but generally the medicament will consist of additional components, such as one or more pharmaceutically acceptable carriers or diluents. The carrier(s) or diluent(s) must be “acceptable” in the sense of not having any deleterious effect on the patient and being compatible with other components of the formulation. The medicament may also contain other therapeutic ingredients having the same or a different therapeutic effect from the agent which inhibits the adhesion of monocytes to oxidised LDL, a component thereof or other lipid raft ligand, for example agents having an effect on the heart or circulation, such as anti-coagulants or antihypertensives.

In the case of small chemical molecules, the agent which inhibits the adhesion of monocytes to oxidised LDL, a component thereof or other lipid raft ligand may be formulated for administration by any suitable means provided that it is delivered to the circulation in such a manner that monocyte adhesion to oxidised LDL, component thereof or other lipid raft ligand in the vicinity of atherosclerotic plaque or at potential sites of atherosclerotic plaque formation can be inhibited.

Examples of suitable forms of administration include oral, parenteral, rectal or intranasal, e.g. by inhalation.

A medicament for oral administration may take the form of, for example, tablets or capsules and may be prepared by processing the agent which inhibits the adhesion of monocytes to oxidised LDL, a component thereof or other lipid raft ligand in a conventional manner together with one or more pharmaceutically acceptable excipients. Tablets may be prepared by compression or moulding in known manner and suitable excipients include binding agents, fillers, lubricants, disintegrants and wetting agents. Tablets or capsules may be coated in known manner, for example to provide slow or controlled release of the active ingredient.

Liquid preparations for oral administration may take the form, for example, of solutions, syrups or suspensions or may be presented as a dry product for re-constitution with water or another suitable vehicle prior to use.

Medicaments for parenteral administration include aqueous and non-aqueous sterile injection solutions which may be formulated in known manner. The formulations may be presented in unit-dose or multi-dose containers, for example, ampoules or vials, or may be stored in a lyophilised condition suitable for reconstitution by addition of sterile liquid, for example water for injection.

Medicaments for rectal administration may be presented in forms such as suppositories or retention enemas which may be formulated in known manner.

Medicaments for intranasal administration may be formulated as solutions for administration via a metered dose or unit device or as a powder including a suitable carrier for administration using an appropriate delivery system.

Antibodies which inhibit monocyte adhesion to oxidised LDL, a component thereof or other lipid raft ligands will generally also be administered to patients in the form of a medicament which preferably includes, in addition to the antibody, a physiologically acceptable carrier or diluent, possibly in admixture with one or more other agents such as other antibodies or drugs, such as antibiotics or agents having an effect on the heart or circulation.

Suitable carriers include physiological saline and phosphate buffered saline. Alternatively the antibody may be lyophilised and reconstituted before use by the addition of an aqueous buffered solution. Routes of administration of the antibody include intravenous, intramuscular, subcutaneous and intraperitoneal injection or delivery.

The method by which the agent which inhibits monocyte adhesion to oxidised LDL, a component thereof or other lipid raft ligand is used in the treatment or prevention of disease will depend on the nature of the agent. Small chemical molecules may be used prophylactically over long periods by subjects at risk of said disease. Antibodies carry more risk of an adverse reaction from the subject's immune system and are more suitable for short term therapy of patients at particular risk in special circumstances, for example risk of atherosclerosis following heart transplantation. In all cases the precise dose to be administered will be at the discretion of the attendant physician but will depend on the nature of the agent and a number of other factors including the age and sex of the patient, the condition of the patient and the severity of the disorder being treated.

### EXAMPLES

The invention is now described further with reference to the following Examples.

### Example 1

### Solid Phase Oxidised LDL is a Potent Adhesion Ligand for Monocytes

LDL is obtained from human serum by density gradient centrifugation, and oxidised with copper sulphate 5 μmol/L, as described by Siow et al (1998)²⁷. Black 96 well plates (Corning) were optimally coated overnight at 4° C. with 50 μL of 10 μg/ml of oxidised LDL or isolated native LDL as a negative control. The native LDL was the material from which the oxidised LDL was derived. Uncoated wells, or bovine serum albumin at the same concentration were also used as negative controls. The wells were then washed three times with PBS and used in the adhesion experiment.

U937 cells were stimulated overnight with PMA 10 ng/ml for 16 hours, and washed three times in RPMI+10% FCS. The U937 cells or monocytes at about 6×10⁶/ml were then incubated with 5 (and 6)-carboxyfluorescein diacetate succinimidyl ester 20-100 μM (C-1157, Molecular Probes) for 30 minutes. This compound allows fluorescein labelling of viable cells. The cells were washed three times in RPMI+10% FCS, and added to the 96 well plates at 3.2×10⁵/well. Assays were done at least in triplicate. The plates were incubated at 37° C. for 40 minutes or 1 hour. The plates were then washed by shaking out the cells and washing three times in a bath of PBS. Aliquots of the original cell suspension were added to at least three unused wells as reference. The plates were then counted in an automatic fluorescent spectrophotometer. The data are expressed as the % of input cells bound ±SD, compared to the reference wells.

As shown in FIG. 1, oxidised LDL induced substantial levels of binding, which was approximately equal to the binding to fibronectin used as the positive control (not shown), whereas native LDL gave very little adhesion. In this figure n=6 for each sample and ***P<0.001 compared with n-LDL+cells.

### Example 2

### Oxidised LDL is Specifically Localised in the Endothelial Cells Over Atherosclerotic Lesions

To determine the possibility of oxidised LDL being a ligand binding monocytes to atherosclerotic plaques, the distribution of LDL and oxidised LDL were investigated by immunohistochemistry (IHC) in atherosclerotic human carotid artery operative specimens. IHC was done by the ABC technique with optimally-diluted antibodies. It was found that the arterial endothelium reacted very strongly and universally for LDL, as determined by both monoclonal and polyclonal antibodies in a panel of 20 arteries. This expression is at a higher level than that in the remainder of the intima. These findings have not previously been reported in the literature. This expression is however not specific to LDL, because IgM gave a similar result, and probably reflects a physiological transport of plasma proteins into the arterial intima.

Nevertheless, the expression of oxidised LDL was determined by IHC with MDA-2, a monoclonal reactive with oxidised LDL epitopes (see FIG. 2). Endothelial expression was detected (endothelium stained for oxidised LDL by ABC immunohistochemistry with MDA2 anti-oxLDL—see arrow in figure), but was much more limited than native LDL, and was related to the presence of atherosclerotic lesions. This finding supports a role in oxidised LDL in monocyte adhesion.

The finding that oxidised LDL itself can induce monocyte adhesion raises the possibility that the oxidised LDL itself presented on the surface of the endothelial cells is an adhesion ligand in these experiments. Alternatively oxidised components derived from oxidised LDL may be presented on the cell surface. In addition, oxidised LDL may be formed from LDL within endothelial cells by oxidant stress, particularly when the cells are activated, as occurs in atherosclerotic plaques.

### Example 3

### Identification of Inhibitors of the Adhesion of Monocytes to Oxidised LDL

Plastic wells were coated with oxidised LDL, or fibronectin 10 μg/ml for 16 hrs at 4° C., and washed. Monocytes were isolated in an unactivated state from one unit of buffy coat cells by the method of Tsouknos et al²⁶, labelled with carboxyfluorescein for 30 mins, and washed. Inhibitors and control Ig UPC10 were added to the plates in 50 μL, and then 50 μL of cells at a final concentration of 3×10⁶/ml in the assay. The plates were incubated at 37° C. for 1 hr. Non-adherent cells were removed by a standardised washing procedure, and after addition of cell suspension to 3 wells as the 100% control, the plates were measured in an automatic fluorescent spectrophotometer.

FIG. 3 shows that MDA2 (anti-malondialdehyde modified lysine in apoproteins of oxidised LDL), anti-LDL and anti-CD14 antibodies all inhibited binding of monocytes to oxidised LDL by approximately 60%.

### Example 4

### Metabolic Inhibitors of the Adhesion of Blood Monocytes to Oxidised LDL

The experiment was performed as in Example 3. Results are shown in FIG. 4, the legend of which is explained below.

Control wells:

nLDL:—well coated with native LDL;

oxLDL:—control well coated with ox-LDL without inhibitors;

HCO3:—uncoated well exposed to coating buffer only.

The inhibitors were added to oxidised LDL coated wells:

EDTA ligates calcium, and blocks integrin mediated adhesion;

polyin:—polyinosine is an inhibitor of type A scavenger receptors;

SB203580 is an inhibitor of p38 MAPkinase;

LY294002 and wortmannin are inhibitors of PI3 kinase;

HA-1077 (fasudil) is an inhibitor of Rho kinase.

All the receptor and signaling inhibitors used gave a marked inhibition of monocyte adhesion to oxidised LDL. P<0.05 for all inhibitors with respect to oxidised LDL.

### Example 5

### Lipid Raft Disruption Inhibits the Adhesion of Monocytes to Oxidised LDL

U937 cells were stimulated with PMA 10 ng/ml, plus vitamin A (1 ug/ml) and vitamin D (10-7 M) for 16 hours, and then the assay was performed as in Example 3. The raft-disrupting agents, nystatin (NST) and methyl cyclodextrin (MCD) caused near-total inhibition of activated U937 cell adhesion to oxidised LDL at all concentrations tested, as shown in FIG. 5.

### Example 6

### Inhibition of Oxidised LDL Adhesion by Murine Immunoglobulin

U937 cells were stimulated as in Example 5, and the experiment performed as in Example 3. The results are shown in FIG. 6, with the legend as in the previous figures. CD14, CD18, TLR-4 antibodies were all murine Igs. MOPC21:—control murine IgG₁, UPC10:—control murine IgG₂ₐ. All murine immunoglobulins, including control non-antibody types, caused marked inhibition of adhesion to ox-LDL of highly activated U937 cells.

### Example 7

### Inhibition of Adhesion of Highly Activated U937 Cells to HSP60 by Human Immunoglobulin

U937 cells were stimulated as in Example 5, and experiment performed as in Example 3, except that wells were coated with HSP60 5 μg/ml. HSP60 is also a CD14/lipid raft ligand. Human polyclonal IgG, and isolated IgG Fc fragments gave significant inhibition of highly activated U937 cell adhesion to HSP60, as shown in FIG. 7. The activity of Fc fragments suggests that Fc receptors play an inhibitory role in lipid rafts.

### Example 8

### Inhibition of the Binding of Highly Activated U937 Cells to Fibronectin by Murine Immunoglobulins

The experiment was performed as in Examples 3 and 6, except that the wells were coated with fibronectin 10 μg/ml. As shown in FIG. 8, all murine Igs, including the non-immune forms MOPC21 and UPC10, caused around 50%-60% inhibition of the adhesion of highly activated U937 cells to fibronectin. Fibronectin is a ligand for integrin adhesion molecules.

### Example 9

### Inhibition of Adhesion of Highly Activated U937 Cells to HSP60 by DMSO

The experiment was performed as in Example 7. Dimethylsulphoxide (DMSO) was tested in HSP60 coated wells; the concentration in the assay is given as dilution from neat. As shown in FIG. 9, DMSO was highly inhibitory to the binding of highly activated U937 cells to HSP60, a lipid raft ligand, at the lowest concentration tested. It is likely that it acts by disrupting the lipid environment of the lipid raft. DMSO also inhibits adhesion of monocytes to tissue sections of atherosclerotic plaque.

### REFERENCES

¹ M C Bourdillon, R N Poston, C Covacho, E Chignier, G Bricca, J L McGregor (2000). ICAM-1 deficiency reduces atherosclerotic lesions in double knockout mice (apo-E −/−/ICAM-1 −/−) fed a fat or a chow diet. Arterioscl Thromb Vasc Biol 20: 2630-2635

² Beekhuizen H, van Furth R. Monocyte adherence to human vascular endothelium. J. Leukoc. Biol. 1993;54(4):363-78

³ Poston R N, Johnson-Tidey R R. Localized adhesion of monocytes to human atherosclerotic plaques demonstrated in vitro: implications for atherogenesis. Am. J. Pathol. 1996;149(1):73-80

⁴ Schmitz G, Orso E. CD14 signaling in lipid rafts: new ligands and co-receptors. Curr. Opin. Lipidol. 2002;13(5):513-21

⁵ Triantafilou M, Miyake K, Golenbock D T, Triantafilou K. Mediators of innate immune recognition of bacteria concentrate in lipid rafts and facilitate lipopolysaccharide-induced cell activation. J. Cell Sci. 2002;115(Pt 12):2603-11

⁶ Leitinger B, Hogg N. The involvement of lipid rafts in the regulation of integrin function. J. Cell Sci. 2002;115(Pt 5):963-72

⁷ van Kooyk Y, Figdor C G. Avidity regulation of integrins: the driving force in leukocyte adhesion. Curr. Opin. Cell Biol. 2000;12(5):542-7

⁸ Kol A, Lichtman A H, Finberg R W, Libby P, Kurt-Jones E A. Cutting edge: heat shock protein (HSP) 60 activates the innate immune response: CD14 is an essential receptor for HSP60 activation of mononuclear cells J. Immunol. 2000;164(1):13-7

⁹ Amberger A, Maczek C, Jurgens G, Michaelis D, Schett G, Trieb K et al. Co-expression of ICAM-1, VCAM-1, ELAM-1 and Hsp60 in human arterial and venous endothelial cells in response to cytokines and oxidized low-density lipoproteins Cell Stress. Chaperones. 1997;2(2):94-103

¹⁰ Poston, R. N., Louis, H., Aijaz, B., Lovett, N., and Taylor, P. R. Heat shock protein 60 mediates monocyte adhesion via CD14: role in the adherence of monocytes to atherosclerotic plaques. Atherosclerosis Suppl 4, 147. 2003

¹¹ WO 03/048783

¹² Ohashi K, Burkart V, Flohe S, Kolb H. Cutting edge: heat shock protein 60 is a putative endogenous ligand of the toll-like receptor-4 complex. J. Immunol. 2000;164(2):558-61

¹³ Solomon K R, Kurt-Jones E A, Saladino R A, Stack A M, Dunn I F, Ferretti M et al. Heterotrimeric G proteins physically associated with the lipopolysaccharide receptor CD14 modulate both in vivo and in vitro responses to lipopolysaccharide. J. Clin. Invest 1998;102(11):2019-27

¹⁴ Schmidt A, Caron E, Hall A. Lipopolysaccharide-induced activation of beta2-integrin function in macrophages requires Irak kinase activity, p38 mitogen-activated protein kinase, and the Rap1 GTPase. Mol. Cell Biol. 2001;21(2):438-48

¹⁵ Bjorkbacka H, Kunjathoor V V, Moore K J, Koehn S, Ordija C M, Lee M A et al. Reduced atherosclerosis in MyD88-null mice links elevated serum cholesterol levels to activation of innate immunity signaling pathways. Nat. Med. 2004;10(4):416-21

¹⁶ Miller Y I, Viriyakosol S, Binder C J, Feramisco J R, Kirkland T N, Witztum J L. Minimally modified LDL binds to CD14, induces macrophage spreading via TLR4/MD-2, and inhibits phagocytosis of apoptotic cells J. Biol. Chem. 2003;278(3):1561-8

¹⁷ Miller Y I, Chang M K, Binder C J, Shaw P X, Witztum J L. Oxidized low density lipoprotein and innate immune receptors. Curr. Opin. Lipidol. 2003;14(5):437-45

¹⁸ Frostegard J, Haegerstrand A, Gidlund M, Nilsson J. Biologically modified LDL increases the adhesive properties of endothelial cells. Atherosclerosis 1991;90(2-3):119-26

¹⁹ Jeng J R, Chang C H, Shieh S M, Chiu H C. Oxidized low-density lipoprotein enhances monocyte-endothelial cell binding against shear-stress-induced detachment Biochim. Biophys. Acta 1993;1178(2):221-7

²⁰ Klouche M, May A E, Hemmes M, Messner M, Kanse S M, Preissner K T et al. Enzymatically modified, nonoxidized LDL induces selective adhesion and transmigration of monocytes and T-lymphocytes through human endothelial cell monolayers. Arterioscler. Thromb. Vasc. Biol. 1999;19(3):784-93

²¹ Dwivedi A, Anggard E E, Carrier M J. Oxidized LDL-mediated monocyte adhesion to endothelial cells does not involve NfkappaB Biochem. Biophys. Res. Commun. 2001;284(1):239-44

²² Bromley S K, Burack W R, Johnson K G, Somersalo K, Sims T N, Sumen C et al. The immunological synapse. Annu. Rev. Immunol. 2001;19:375-96

²³ J. Leukocyte Biol., 37, 407-422

²⁴ Int. J. Cancer, 17, 565-577

²⁵ Int. J. Cancer, 26, 171

²⁶ Tsouknos A, Nash G B, Rainger G E. Monocytes initiate a cycle of leukocyte recruitment when cocultured with endothelial cells. Atherosclerosis 2003;170(1):49-58

²⁷ Siow R C, Sato H, Leake D S, Pearson J D, Bannai S, Mann G E. Vitamin C protects human arterial smooth muscle cells against atherogenic lipoproteins: effects of antioxidant vitamins C and E on oxidized LDL-induced adaptive increases in cystine transport and glutathione. Arterioscler. Thromb. Vasc. Biol. 1998;18(10):1662-70

²⁸ Zacharias D A, Violin J D, Newton A C, Tsien R Y. Partitioning of lipid-modified monomeric GFPs into membrane microdomains of live cells. Science 2002;296(5569):913-6

1. A method for identifying agents which inhibit binding of monocytes to oxidised LDL or a component thereof which comprises:
contacting a suspension of monocytes with immobilised oxidised LDL or a component thereof, in the presence and absence of a potential inhibitory agent, or with and without preincubation of the monocyte suspension or immobilised oxidised LDL or component thereof with a potential inhibitory agent;
quantitating the number of bound monocytes over a defined area of oxidised LDL or component thereof; and
assessing the effect of the agent on the extent of monocyte binding, wherein agents which inhibit binding of monocytes to oxidised LDL or component thereof are identified by a reduction in the number of bound monocytes over the defined area.

2. The method according to claim 1 wherein an agent which inhibits binding of monocytes to oxidised LDL or component thereof is identified by a 50% reduction in the number of bound monocytes over the defined area.

3. The method according to claim 1 or 2 wherein the oxidised LDL or component thereof is immobilised on the surface of a plastic plate.

4. The method according to any of claims 1 to 3 wherein the component of oxidised LDL is selected from oxidised apoproteins, oxidised phospholipids and oxidised neutral lipids.

5. A method for the inhibition of monocyte adhesion to oxidised LDL or component thereof in a patient which comprises administering to the patient an effective amount of an agent which inhibits said monocyte adhesion to oxidised LDL or component thereof.

6. The method according to claim 5 wherein the component of oxidised LDL is selected from oxidised apoproteins, oxidised phospholipids and oxidised neutral lipids.

7. A method for the inhibition of lipid raft mediated monocyte adhesion in a patient which comprises administering to the patient an effective amount of an agent which inhibits said lipid raft mediated monocyte adhesion.

8. A method according to any of claims 5 to 7 wherein the agent produces a reduction of at least 50% in the number of bound monocytes in a method as defined in claim 1.

9. A method according to any of claims 5 to 8 wherein said inhibition is for the therapy or prophylaxis of atherosclerosis.

10. A method according to any of claims 5 to 8 wherein said inhibition is for the therapy or prophylaxis of rheumatoid arthritis, multiple sclerosis or glomerulonephritis.

11. A method according to any of claims 5 to 10 wherein the agent is nystatin, methyl cyclodextrin, dimethylsulphoxide, or combinations or derivatives thereof.

12. A method according to any of claims 5 to 10 wherein the agent is an immunoglobulin, a signaling pathway inhibitor, or a ligand at monocyte immunoglobulin receptors, preferably at the lipid raft-related Fc receptors CD16, CD32 or CD64.

13. A method according to claim 12 wherein the agent is polyinosine, SB203580, LB294002, wortmannin, HA-1077, or combinations or derivatives thereof.

14. Use of an agent that inhibits the adhesion of monocytes to oxidised LDL or a component thereof for the manufacture of a medicament for the therapy or prophylaxis of a condition involving monocyte adhesion to oxidised LDL or component thereof by a method wherein monocyte adhesion to oxidised LDL or component thereof is inhibited.

15. Use according to claim 14 wherein the component of oxidised LDL is selected from oxidised apoproteins, oxidised phospholipids and oxidised neutral lipids.

16. Use of an agent that inhibits the lipid raft mediated monocyte adhesion for the manufacture of a medicament for the therapy or prophylaxis of a condition involving lipid raft mediated monocyte adhesion by a method wherein said lipid raft mediated monocyte adhesion is inhibited.

17. Use according to any of claims 14 to 16 wherein the agent produces a reduction of at least 50% in the number of bound monocytes in a method as defined in claim 1.

18. Use according to any of claims 14 to 17 wherein the medicament is for the therapy or prophylaxis of atherosclerosis.

19. Use according to any of claims 14 to 17 wherein the medicament is for the therapy or prophylaxis of rheumatoid arthritis, multiple sclerosis or glomerulonephritis.

20. Use according to any of claims 14 to 19 wherein the agent is nystatin, methyl cyclodextrin, dimethylsulphoxide, or combinations or derivatives thereof.

21. Use according to any of claims 14 to 19 wherein the agent is an immunoglobulin, a signaling pathway inhibitor, or a ligand at monocyte immunoglobulin receptors, preferably at the lipid raft-related Fc receptors CD16, CD32 or CD64.

22. Use according to claim 21 wherein the agent is polyinosine, SB203580, LB294002, wortmannin, HA-1077, or combinations or derivatives thereof.
